(19) United States
(12) Patent Application Publication
(10) Pub. No.: US 2022/0144558 A1
(43) Pub. Date: May 12, 2022

(54) APPARATUS FOR AUTOMATED APPLICATION OF A SOLUTION ONTO A CONVEYOR BELT

(71) Applicant: FRESHBELT SYSTEMS LTD., Surrey (CA)

(72) Inventors: Jamie Mackay, Surrey (CA); Michael McAdam, Surrey (CA); Trevor Hein, Surrey (CA)

(21) Appl. No.: 17/510,183

(22) Filed: Oct. 25, 2021

Related U.S. Application Data
(60) Provisional application No. 63/104,746, filed on Oct. 23, 2020.

(51) Int. Cl. B65G 45/22; B65G 45/12

(52) U.S. Cl. CPC B65G 45/22; B65G 45/12

(57) ABSTRACT

Checkout conveyor belts can become contaminated with a variety of bacteria and pathogens. Manual disinfection of the conveyor belt is time consuming and results in excess wetness. The invention is an apparatus for automated application of a disinfectant solution to a checkout conveyor belt. The apparatus includes an elongate applicator, a contact surface, an internal elongate cavity, and a solution delivery assembly. The applicator mounts to an end of the belt housing and spans the width of the belt. The contact surface contacts the outward facing surface of the belt. A dispensing tube evenly distributes a flow of disinfectant solution to the cavity of the applicator adjacent the contact surface. The solution delivery assembly delivers a flow of disinfectant solution to the dispensing tube. A cover assembly urges the contact surface of the applicator against the outward facing surface of the belt and covers non-belt contacting surfaces of the applicator.

### BACKGROUND OF INVENTION

### 1. Field of the Invention

The invention relates generally to apparatus for applying a solution to a conveyor belt and more specifically to an apparatus for automated application of a disinfectant solution onto a retail checkout conveyor belt.

### 2. Description of the Related Art

Many retail stores use loop conveyor belts at checkout counters to move retail items from an end near where the belt emerges from its belt housing toward the other belt end where the belt recedes into its housing. The outward facing surface upon which the retail items rest as they are conveyed can become dirty and contaminated with pathogens and may provide a mode by which these pathogens can be physically communicated to items or persons who come into contact with the contaminated belt. This can particularly be a major problem when food items are being placed on the conveyor belts. However, most stores or other facilities may be unable or unwilling to manually clean conveyor belts between customers as this slows the checkout process which could result in inconvenience to the customers as well as a loss of productivity and profits to the store. And even if the conveyor belts are halted for cleaning, the cleaning process involves manual intervention which may be inconvenient to the checkout staff operating the checkout counter and may result in insufficient cleaning of the conveyor belt. While automated conveyor belt cleaning apparatus have been developed to address these problems, many of the automated conveyor belt apparatus of the prior art suffer from one or more of complexity, cost, lack of being able to be retrofitted to existing checkout apparatus, reliance on unproven technology, impracticality, or may involve a risk of unnecessary unsafe exposure of disinfectant solutions to customers. Accordingly, there exists a need for an improved apparatus for automated application of a disinfectant solution onto conveyor belts, particularly for loop conveyor belts at retail store checkout counters.

### SUMMARY OF THE INVENTION

In an aspect, the present invention provides an apparatus for automated application of a disinfectant solution onto a retail checkout conveyor belt moving in a circular loop and having a width and an outward facing surface upon which retail items are conveyed and a belt end where the belt recedes into a housing around a roller, the apparatus comprising: an elongate applicator having ends, a contact surface, and defining an internal elongate cavity extending between the ends, the applicator being configured to span the width of the belt; a mounting bracket cooperating with each end of the applicator to removably secure each end to the housing near the belt end in a manner that the applicator spans the width of the belt and the contact surface is in contact with the outward facing surface of the belt; a dispensing tube received within the elongate cavity and having small openings along its length to evenly distribute a flow of solution to the cavity of the applicator adjacent the contact surface; a solution delivery assembly operable to deliver the flow of solution to the dispensing tube; and a cover assembly that urges the contact surface of the applicator against the outward facing surface of the belt and that covers non-belt contacting surfaces of the applicator.

In some embodiments, the contact surface of the applicator may comprise cotton chamois. In some embodiments, the entire applicator may comprise cotton chamois.

In some embodiments, the applicator may have an element width and an element height, and the element width is at least twice the element height to provide a low profile to the applicator.

In some embodiments, the cover assembly may comprise a cover plate that conforms to the dimensions of the applicator. In some embodiments, the cover plate may define a leading edge operable to deflect retail items on the belt from the applicator as the belt moves past the applicator.

In some embodiments, the cover assembly may further comprise a planar enclosure member connected to the cover plate and being configured to cooperate with the belt end to enclose the belt end.

In some embodiments, the cover assembly may further comprise a hinge mechanism connected to the enclosure member and that cooperates with the housing to enable the enclosure member and the cover plate to move from a closed position in which the cover plate covers the applicator and the enclosure member encloses the housing to an open position in which the applicator and the housing are exposed.

In some embodiments, the cover assembly may further comprise a mounting plate on which the hinge mechanism is connected, the mounting plate being configured for attachment to an inside wall of the housing in a manner to enable movement of the cover plate and enclosure member to and from the open position and the closed position.

In some embodiments, the solution delivery assembly may comprise a fluid conduit coupled to the dispensing tube, a pump coupled to the fluid conduit, and a solution source, wherein the pump is operable to provide the flow of solution from the solution source to the dispensing tube.

In some embodiments, the solution source may comprise a suction conduit coupled to an inlet of the pump, a solution bottle to hold the solution and to receive the suction conduit, and a removable bottle cap adapted to seal the solution bottle against outflow of solution other than through the suction conduit.

In some embodiments, activation of the pump may be synchronized with movement of the belt in a manner that the pump provides a continuous flow of solution to the applicator while the belt is in motion. In some embodiments, the pump may be electrically powered and its synchronization with movement of the belt comprises electrically coupling the pump to an electric circuit that provides electricity to a motor for driving the conveyor belt.

In some embodiments, the solution delivery assembly may further comprise a shut off valve downstream of the pump to enable an operator to manually stop the flow of solution to the dispensing tube.

In some embodiments, the applicator may further comprise a tab at each end and the mounting bracket is operable to grasp the tab to secure the tab to the belt housing.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 is a perspective partial view from a side of an example of a typical retail checkout conveyor belt and housing;

FIG. 2 is a perspective partial side view of an embodiment of an apparatus for automated application of a disinfectant solution onto a retail checkout conveyor belt in accordance with the present invention;

FIG. 3 is an exploded view of a portion of the applicator, cover assembly and a portion of the solution delivery assembly of the apparatus shown in FIG. 2;

FIG. 4 is a perspective view of the applicator of the apparatus shown in FIG. 2;

FIG. 5 is a perspective view of the apparatus of FIG. 2 with the cover assembly removed showing the applicator mounted on the belt housing;

FIG. 6 is a perspective view of the apparatus of FIG. 2 with the cover assembly removed showing the applicator mounted on the belt housing, and the cavity portion of the applicator shown in phantom revealing the fluid dispensing tube within the internal cavity of the applicator;

FIG. 7 is a closeup side view of a portion of the solution delivery assembly of the apparatus shown in FIG. 2 showing the bottle and pump mechanism; and

FIG. 8 is a longitudinal section of the belt housing and apparatus of FIG. 2.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

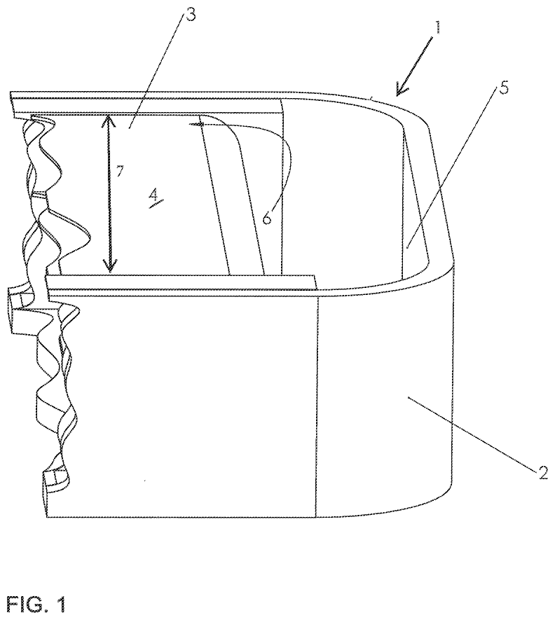

In FIG. 1 there is shown a partial view of an example of a typical retail checkout conveyor belt system showing belt end 1 of the belt housing 2, loop conveyor belt 3 having an outer facing surface 4 upon which retail items are conveyed. At the belt end 1 the belt 3 emerges from the housing 2 around a roller 8. Also shown is end wall 5 of the housing 2. The conveyor belt 3 typically travels in direction shown by arrow 6.

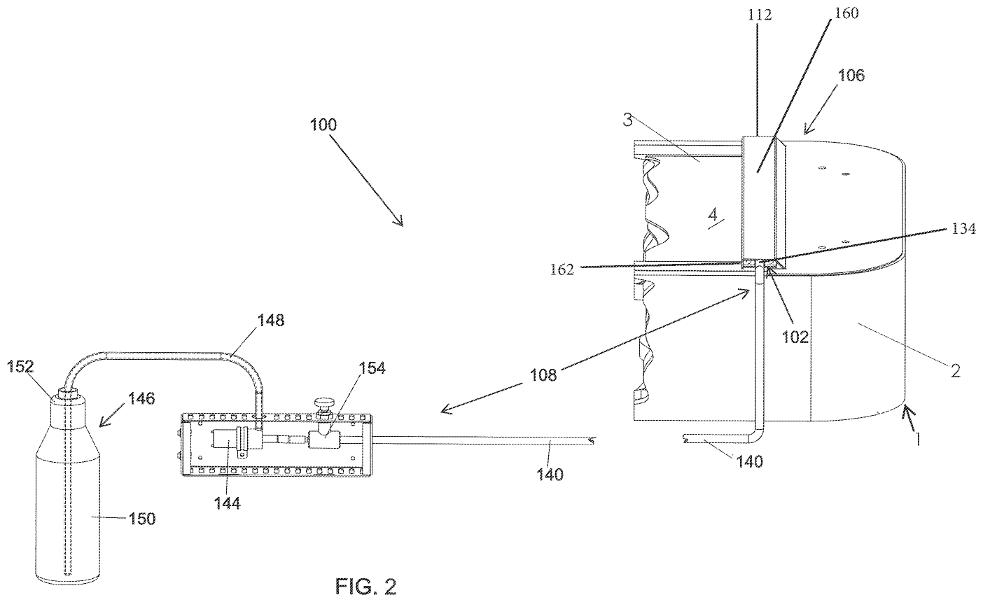

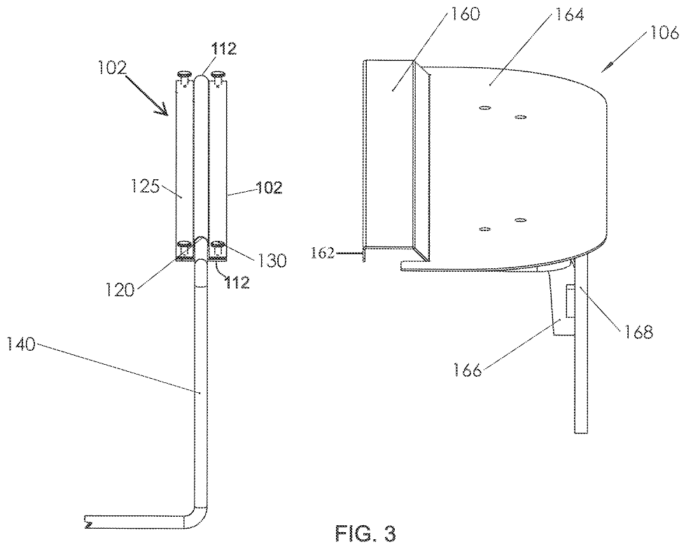

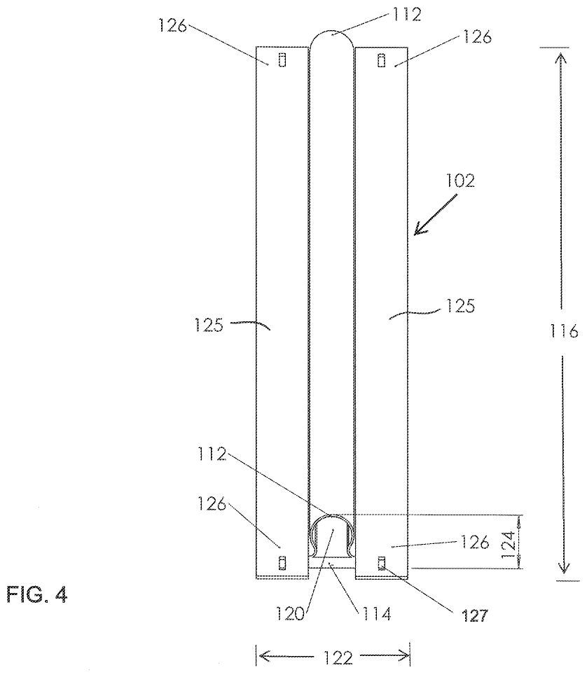

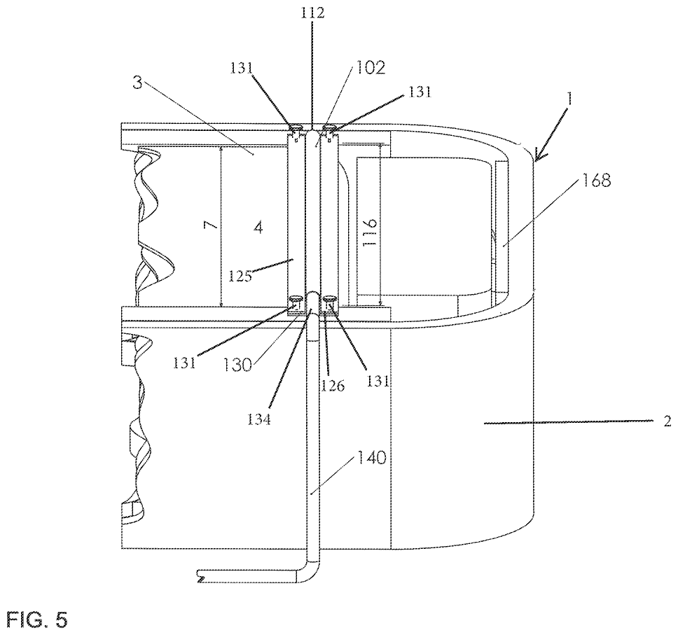

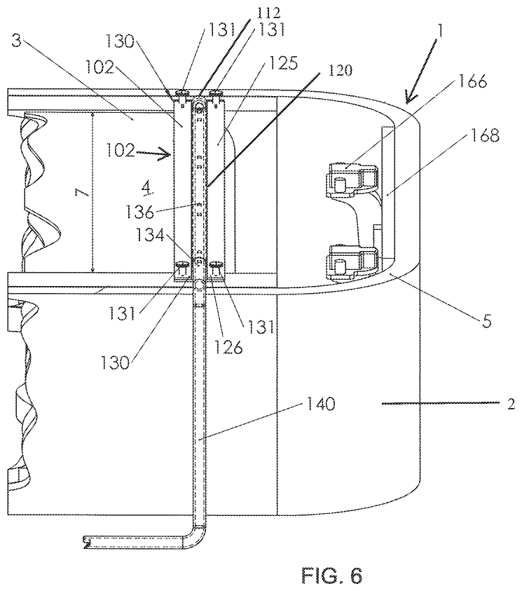

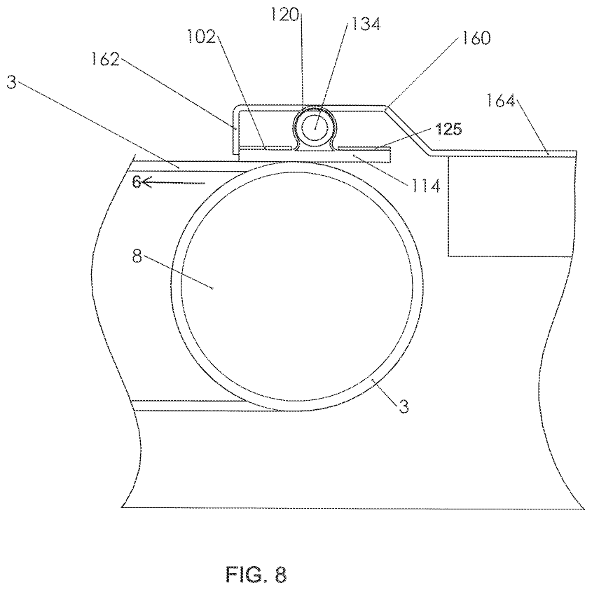

Referring to FIGS. 2-8, there is shown an apparatus for automated application of a disinfectant solution 100 on a retail checkout conveyor belt in accordance with an embodiment of the present invention. Automated solution application apparatus 100 includes applicator 102, cover assembly 106 and cleaning/disinfectant solution delivery assembly 108. In general, a flow of cleaning/disinfectant solution is provided to the applicator 102 by the solution delivery assembly 108 to wet the applicator with the solution. The cover assembly 106 encloses the applicator 102 and urges it against the outer facing surface 4 of the belt 3. As the belt 3 moves past the applicator 102, the outer facing surface 4 is cleaned and/or disinfected.

Applicator 102 may be an elongate applicator having ends 112 and a contact surface 114 that is maintained in contact with outward facing surface 4 of the conveyor belt 3. In its length 116, applicator 102 preferably spans the width 7 of the conveyor belt 3 so that the entire width of the outer surface 4 is cleaned and/or disinfected as it slides past the contact surface 114 of the applicator 102.

Applicator 102 defines an internal elongate cavity 120 extending between the ends 112. Preferably, the applicator 102 has a low profile such that its width 122 is at least twice its height 124. Applicator 102 includes an elongate mounting strip 125 on each side of the cavity 120 and extending the length 116 of the applicator 102. The elongate mounting strip 125 may comprise a strip of a durable fabric material, such as nylon webbing or the like, that is attached to the applicator. Alternatively, the elongate mounting strip 125 may comprise a strip of more rigid material such as a thin metal, thermoplastic and the like. The elongate mounting strip 125 includes mounting tabs 126 at each end by which the applicator 102 is mounted to the housing 2 near the belt end 1 by mounting brackets 130.

Contact surface 114 of the applicator 102 is preferably made of cotton chamois. The entire applicator 102 may be made of cotton chamois in which case the elongate mounting strips 125 may comprise the chamois material folded over itself to form a hem that provides the requisite support. In other embodiments, the applicator 102 may comprise microfiber material or similar material.

With particular reference to FIGS. 2, 5 and 6, automated solution application apparatus 100 includes mounting brackets 130, each cooperating with an end 112 of the applicator 102 to removably secure the end 112 to the housing 2 near the belt end in a manner that the applicator 102 spans the width 7 of the belt 3 and the contact surface 114 is in contact with the outward facing surface 4 of the belt 3. The mounting brackets 130 can be configured by an operator to release the applicator 102 so that a worn applicator 102 may be replaced with a new applicator.

Mounting tabs 126 facilitate mounting of the applicator 102 onto the housing 2 by the mounting brackets 130. The mounting bracket 130 removably grasps the tab 126 to secure the tab to the belt housing and thereby securely mount the applicator 102 to the housing. The mounting bracket 130 can release the tabs 126 upon input from a user. More specifically, mounting bracket 130 comprises a screw 131 that passes through a slot 127 in each tab 126 to secure the tab to the housing. The elongate mounting strips 125 are configured to be taut when the tabs 126 at each end are mounted to the housing and thereby provide additional strength and rigidity to the mounted applicator 102 to resist deformation thereof as a result of friction caused by the moving belt 3.

With reference to FIGS. 2, 5 and 6, automated solution application apparatus 100 includes a drip line or fluid dispensing tube 134 within the internal cavity 120 of the applicator 102, preferably spanning the length of the elongate cavity 120. Dispensing tube 134 includes openings or perforations 136 along its length to evenly distribute by gravity a flow of cleaning/disinfecting solution to the cavity 120 of the applicator, particularly to a portion of the cavity adjacent the contact surface 114 so that the contact surface 114 is evenly wetted with cleaning/disinfecting solution. Advantageously, the drip line dispenses the solution into the applicator 102 that controls the amount of solution on the belt, eliminating over spray that is common with some prior art devices that rely on a solution spray.

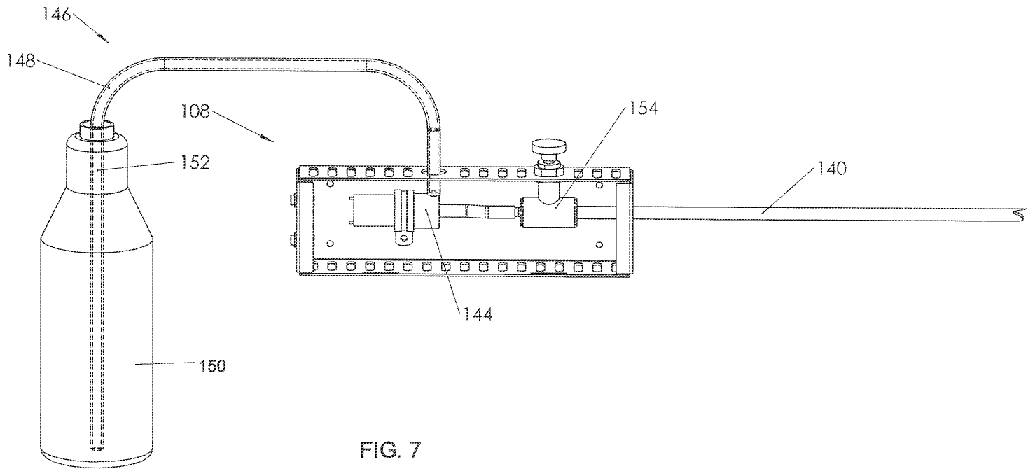

With reference to FIGS. 2 and 7, automated solution application apparatus 100 includes cleaning/disinfecting solution delivery assembly 108 that includes a cleaning/disinfecting solution conduit such as fluid conduit 140 coupled to the dispensing tube 134, a pump 144 coupled to the fluid conduit 140, and a solution source 146 such that the pump 144 is operable to provide the flow of solution from the solution source 146 to the dispensing tube 134. The solution source 146 includes a suction conduit 148 coupled to an inlet of the pump 144, a solution bottle 150 to hold the cleaning/disinfecting solution and to receive the suction conduit 148, and a removable bottle cap or plug 152 adapted to seal the solution bottle 150 against outflow of solution other than through the suction conduit 148. Advantageously, the bottle cap or plug 152 may be configured to fit commonly used cleaning/disinfecting solution bottles that are commercially available. Accordingly, an empty solution bottle 150 can be easily replaced with a full bottle by simply unscrewing the bottle from the cap 152 and screwing on the replacement full bottle.

A shut off valve 154 may be provided downstream of the pump 144 to enable an operator to manually stop the flow of solution to the dispensing tube 134.

Preferably, the activation of the pump 144 is synchronized with movement of the belt 3 such that the pump provides a continuous flow of cleaning/disinfecting solution to the applicator 102 while the belt is in motion. The synchronization may be achieved by a physical coupling between the pump and the belt. Preferably the pump 144 is electrically powered and its synchronization with movement of the belt is accomplished by electrically connecting the pump 144 to an electric circuit that provides electricity to a motor for driving the conveyor belt 3.

In some embodiments, the pump 144 is provided with a timer control that enables adjustment of the duration of time between activation of the pump as well as the amount of time the system pumps the fluid. This allows for a higher pressure of fluid flowing out of the holes but helps control the amount of liquid that gets on the applicator 102. For example, a timer control may include a timer located inside the control and electrically coupled to the pump.

Accordingly, the conveyor belt automated solution application apparatus 100 may be used for the automated cleaning and disinfecting of a conveyor belt 3 at a checkout station of a retail store during its use. The automated solution application apparatus 100 may allow for the conveyor belt 3 to be automatically cleaned and disinfected as it rotates during normal operation, and may rely on the operation of the conveyor belt itself for performing cleaning and/or disinfecting actions, and may be installed into an existing conveyor belt station, or any conveyor belt station or the conveyor system may be constructed with the automated solution application apparatus 100 integrated therein. Thus, an advantage may be that no user interaction is needed for the conveyor belt to be cleaned and/or disinfected.

With reference to FIGS. 1, 2, 3, and 4, automated solution application apparatus 100 includes cover assembly 106 that urges the applicator 102, hence contact surface 114, against the outward facing surface 4 of the belt 3 and that covers the other non-belt contacting surfaces of the applicator 102. Cover assembly 106 includes a cover plate 160 that conforms to the dimensions of the applicator 102 and is operable to urge the applicator 102 into contact with the outward facing surface 4 of the belt 3 thereby providing an even distribution of the cleaner/disinfectant on the surface. Cover plate 160 may include an edge flange 162 operable to deflect retail items from the applicator 102. A planar enclosure member 164 is connected to the cover plate 160 and is configured to cooperate with the belt end 1 to enclose the belt end (as shown in FIG. 2).

Depending on the configuration of the conveyor belt housing onto which the automated solution application apparatus 100 is to be mounted, the cover assembly 106 may include a hinge mechanism such as hinges 166 connected to the enclosure member 164 and that cooperate with the end wall 5 of the housing 2 to enable the enclosure member 164 together with the cover plate 160 to move from a closed position in which the cover plate 160 covers the applicator 102 and the enclosure member 164 encloses the housing 2 (as shown in FIG. 2) to an open position in which the applicator 102 and the inside of the housing are exposed. Cover assembly 106 may include a mounting plate 168 on which the hinge mechanism 166 may be connected, and that may be configured to being mounted to an inside of end wall 5 of the housing 2 in a manner to enable hinged movement of the cover plate and enclosure member to and from the open position and the closed position. The hinges and mounting plate may be used where it is desirable to have the enclosure member be hingedly connected to the housing 2 to enable access to the applicator 102 for maintenance. In other embodiments the enclosure member may be configured to be removable coupled to the housing in a manner that it enclosed the internals of the housing but may be removed therefrom to expose the applicator 102 for maintenance.

While embodiments of the invention have been described and illustrated, such embodiments should be considered illustrative of the invention only. The invention may include variants not described or illustrated herein in detail. Thus, the embodiments described and illustrated herein should not be considered to limit the invention.

What is claimed is:

1. An apparatus for automated application of a disinfectant solution onto a retail checkout conveyor belt moving in a circular loop and having a width and an outward facing surface upon which retail items are conveyed and a belt end where the belt recedes into a housing around a roller, the apparatus comprising:
an elongate applicator having ends, a contact surface, and defining an internal elongate cavity extending between the ends, the applicator being configured to span the width of the belt;
a mounting bracket cooperating with each end of the applicator to removably secure each end to the housing near the belt end in a manner that the applicator spans the width of the belt and the contact surface is in contact with the outward facing surface of the belt;
a dispensing tube received within the elongate cavity and having openings along its length to evenly distribute a flow of solution to the cavity of the applicator adjacent the contact surface;
a solution delivery assembly operable to deliver the flow of solution to the dispensing tube; and
a cover assembly that urges the contact surface of the applicator against the outward facing surface of the belt and that covers non-belt contacting surfaces of the applicator.

2. The apparatus as claimed in claim 1 wherein the applicator has an element width and an element height, and the element width is at least twice the element height to provide a low profile to the applicator.

3. The apparatus as claimed in any one of claims 1-2 wherein the cover assembly comprises a cover plate that conforms to the dimensions of the applicator.

4. The apparatus as claimed in any one of claims 1-3 wherein the applicator comprises a strip of cloth material in between two elongate mounting strips.

5. The apparatus as claimed in claim 4 wherein each mounting strip includes a tab located at each end that are grasped by the mounting brackets to secure the applicator to the housing.

6. The apparatus as claimed in any one of claims 1-5 wherein the cover assembly further comprises a planar enclosure member connected to the cover plate and being configured to cooperate with the belt end to enclose the belt end.

7. The apparatus as claimed in any one of claims 1-6 wherein the cover assembly further comprises a hinge mechanism connected to the enclosure member and that cooperates with the housing to enable the enclosure member and the cover plate to move from a closed position in which the cover plate covers the applicator and the enclosure member encloses the housing to an open position in which the applicator and the housing are exposed.

8. The apparatus as claimed in claim 7 wherein the cover assembly further comprises a mounting plate on which the hinge mechanism is connected, the mounting plate being configured for attachment to an inside wall of the housing in a manner to enable movement of the cover plate and enclosure member to and from the open position and the closed position.

9. The apparatus as claimed in any one of claims 1-8 wherein the solution delivery assembly comprises a fluid conduit coupled to the dispensing tube, a pump coupled to the fluid conduit, and a solution source, wherein the pump is operable to provide the flow of solution from the solution source to the dispensing tube.

10. The apparatus as claimed in claim 9 wherein the solution source comprises a suction conduit coupled to an inlet of the pump, a solution bottle to hold the solution and to receive the suction conduit, and a removable bottle cap adapted to seal the solution bottle against outflow of solution other than through the suction conduit.

11. The apparatus as claimed in any one of claims 9-10 wherein activation of the pump is synchronized with movement of the belt in a manner that the pump provides a continuous flow of solution to the applicator while the belt is in motion.

12. The apparatus as claimed in claim 11 wherein the pump is electrically powered and its synchronization with movement of the belt comprises electrically coupling the pump to an electric circuit that provides electricity to a motor for driving the conveyor belt.

13. The apparatus as claimed in any one of claims 1-12 wherein the solution delivery assembly further comprises a shut off valve downstream of the pump to enable an operator to manually stop the flow of solution to the dispensing tube.
